(19) United States
(12) Patent Application Publication
(10) Pub. No.: US 2005/0272738 A1
(43) Pub. Date: Dec. 8, 2005

(54) Combined treatment with oxaliplatin and an epidermal growth factor receptor kinase inhibitor

(76) Inventors: Jianping Chen, Nutley, NJ (US); Brian Higgins, Nutley, NJ (US); Kenneth Kolinsky, Nutley, NJ (US)

Correspondence Address: OSI PHARMACEUTICALS, INC., 58 SOUTH SERVICE ROAD, MELVILLE, NY, 11747, US

(21) Appl. No.: 11/145,091

(22) Filed: Jun. 3, 2005

Related U.S. Application Data
(60) Provisional application No. 60/576,435, filed on Jun. 3, 2004.

(51) Int. Cl. A61K 31/498; A61K 31/28

(52) U.S. Cl. 514/249; 514/492

(57) ABSTRACT

The present invention provides a method for treating tumors or tumor metastases in a patient, comprising administering to the patient simultaneously or sequentially a therapeutically effective amount of an EGFR kinase inhibitor and oxaliplatin combination, with or without additional agents or treatments, such as other anti-cancer drugs or radiation therapy. The invention also encompasses a pharmaceutical composition that is comprised of an EGFR kinase inhibitor and oxaliplatin combination in combination with a pharmaceutically acceptable carrier. A preferred example of an EGFR kinase inhibitor that can be used in practicing this invention is the compound erlotinib HCl (also known as Tarceva™).

### CROSS-REFERENCE TO RELATED APPLICATIONS

This application claims the benefit of U.S. Provisional Application No. 60/576,435, filed Jun. 3, 2004, which is herein incorporated by reference in its entirety.

### BACKGROUND OF THE INVENTION

The present invention is directed to compositions and methods for treating cancer patients. In particular, the present invention is directed to combined treatment of patients with oxaliplatin and an epidermal growth factor receptor (EGFR) kinase inhibitor.

Cancer is a generic name for a wide range of cellular malignancies characterized by unregulated growth, lack of differentiation, and the ability to invade local tissues and metastasize. These neoplastic malignancies affect, with various degrees of prevalence, every tissue and organ in the body. A multitude of therapeutic agents have been developed over the past few decades for the treatment of various types of cancer. The most commonly used types of anticancer agents include: DNA-alkylating agents (e.g., cyclophosphamide, ifosfamide), antimetabolites (e.g., methotrexate, a folate antagonist, and 5-fluorouracil, a pyrimidine antagonist), microtubule disrupters (e.g., vincristine, vinblastine, paclitaxel), DNA intercalators (e.g., doxorubicin, daunomycin, cisplatin), and hormone therapy (e.g., tamoxifen, flutamide).

Colorectal cancer is among the leading causes of cancer-related morbidity and mortality in the U.S. Treatment of this cancer depends largely on the size, location and stage of the tumor, whether the malignancy has spread to other parts of the body (metastasis), and on the patient's general state of health. Options include surgical removal of tumors for early stage localized disease, chemotherapy and radiotherapy. However, chemotherapy is currently the only treatment for metastatic disease. 5-fluorouracil is currently the most effective single-agent treatment for advanced colorectal cancer, with response rates of about 10%. Additionally, new agents such as the topoisomerase I inhibitor irinotecan (CPT11), the platinum-based cytotoxic agent oxaliplatin (e.g. Eloxatin™), and the EGFR kinase inhibitor erlotinib ([6,7-bis(2-methoxyethoxy)-4-quinazolin-4-yl]-(3-ethynylphenyl)amine, e.g. erlotinib HCl, Tarceva™) have shown promise in treatment.

Over-expression of the epidermal growth factor receptor (EGFR) kinase, or its ligand TGF-alpha, is frequently associated with many cancers, including breast, lung, colorectal and head and neck cancers (Salomon D. S., et al. (1995) Crit. Rev. Oncol. Hematol. 19:183-232; Wells, A. (2000) Signal, 1:4-11), and is believed to contribute to the malignant growth of these tumors. A specific deletion-mutation in the EGFR gene has also been found to increase cellular tumorigenicity (Halatsch, M-E. et al. (2000) J. Neurosurg. 92:297-305; Archer, G. E. et al. (1999) Clin. Cancer Res. 5:2646-2652). Activation of EGFR stimulated signaling pathways promote multiple processes that are potentially cancer-promoting, e.g. proliferation, angiogenesis, cell motility and invasion, decreased apoptosis and induction of drug resistance. The development for use as anti-tumor agents of compounds that directly inhibit the kinase activity of the EGFR, as well as antibodies that reduce EGFR kinase activity by blocking EGFR activation, are areas of intense research effort (de Bono J. S. and Rowinsky, E. K. (2002) Trends in Mol. Medicine 8:S19-S26; Dancey, J. and Sausville, E. A. (2003) Nature Rev. Drug Discovery 2:92-313). Several studies have demonstrated or disclosed that some EGFR kinase inhibitors can improve tumor cell or neoplasia killing when used in combination with certain other anti-cancer or chemotherapeutic agents or treatments (e.g. Raben, D. et al. (2002) Semin. Oncol. 29:37-46; Herbst, R. S. et al. (2001) Expert Opin. Biol. Ther. 1:719-732; Magne, N et al. (2003) Clin. Can. Res. 9:4735-4732; Magne, N. et al. (2002) British Journal of Cancer 86:819-827; Torrance, C. J. et al. (2000) Nature Med. 6:1024-1028; Gupta, R. A. and DuBois, R. N. (2000) Nature Med. 6:974-975; Tortora, et al. (2003) Clin. Cancer Res. 9:1566-1572; Solomon, B. et al (2003) Int. J. Radiat. Oncol. Biol. Phys. 55:713-723; Krishnan, S. et al. (2003) Frontiers in Bioscience 8, e1-13; Huang, S et al. (1999) Cancer Res. 59:1935-1940; Contessa, J. N. et al. (1999) Clin. Cancer Res. 5:405-411; Li, M. et al. Clin. (2002) Cancer Res. 8:3570-3578; Ciardiello, F. et al. (2003) Clin. Cancer Res. 9:1546-1556; Ciardiello, F. et al. (2000) Clin. Cancer Res. 6:3739-3747; Grunwald, V. and Hidalgo, M. (2003) J. Nat. Cancer Inst. 95:851-867; Seymour L. (2003) Current Opin. Investig. Drugs 4(6):658-666; Khalil, M. Y. et al. (2003) Expert Rev. Anticancer Ther. 3:367-380; Bulgaru, A. M. et al. (2003) Expert Rev. Anticancer Ther. 3:269-279; Dancey, J. and Sausville, E. A. (2003) Nature Rev. Drug Discovery 2:92-313; Kim, E. S. et al. (2001) Current Opinion Oncol. 13:506-513; Arteaga, C. L. and Johnson, D. H. (2001) Current Opinion Oncol. 13:491-498; Ciardiello, F. et al. (2000) Clin. Cancer Res. 6:2053-2063; Patent Publication Nos: U.S. 2003/0108545; U.S. 2002/0076408; and U.S. 2003/0157104; and International Patent Publication Nos: WO 99/60023; WO 01/12227; WO 02/055106; WO 03/088971; WO 01/34574; WO 01/76586; WO 02/05791; and WO 02/089842).

An anti-neoplastic drug would ideally kill cancer cells selectively, with a wide therapeutic index relative to its toxicity towards non-malignant cells. It would also retain its efficacy against malignant cells, even after prolonged exposure to the drug. Unfortunately, none of the current chemotherapies possess such an ideal profile. Instead, most possess very narrow therapeutic indexes. Furthermore, cancerous cells exposed to slightly sub-lethal concentrations of a chemotherapeutic agent will very often develop resistance to such an agent, and quite often cross-resistance to several other antineoplastic agents as well.

Thus, there is a need for more efficacious treatment for neoplasia and other proliferative disorders. Strategies for enhancing the therapeutic efficacy of existing drugs have involved changes in the schedule for their administration, and also their use in combination with other anticancer or biochemical modulating agents. Combination therapy is well known as a method that can result in greater efficacy and diminished side effects relative to the use of the therapeutically relevant dose of each agent alone. In some cases, the efficacy of the drug combination is additive (the efficacy of the combination is approximately equal to the sum of the effects of each drug alone), but in other cases the effect is synergistic (the efficacy of the combination is greater than the sum of the effects of each drug given alone). For example, when combined with 5-FU and leucovorin, oxaliplatin exhibits response rates of 25-40% as first-line treatment for colorectal cancer (Raymond, E. et al. (1998) Semin Oncol. 25(2 Suppl. 5):4-12).

However, there remains a critical need for improved treatments for colorectal and other cancers. This invention provides anti-cancer combination therapies that reduce the dosages for individual components required for efficacy, thereby decreasing side effects associated with each agent, while maintaining or increasing therapeutic value. The invention described herein provides new drug combinations, and methods for using drug combinations in the treatment of colorectal and other cancers.

### SUMMARY OF THE INVENTION

The present invention provides a method for treating tumors or tumor metastases in a patient, comprising administering to the patient simultaneously or sequentially a therapeutically effective amount of an EGFR kinase inhibitor and oxaliplatin combination, with or without additional agents or treatments, such as other anti-cancer drugs or radiation therapy.

The invention also encompasses a pharmaceutical composition that is comprised of an EGFR kinase inhibitor and oxaliplatin combination in combination with a pharmaceutically acceptable carrier.

A preferred example of an EGFR kinase inhibitor that can be used in practicing this invention is the compound erlotinib HCl (also known as Tarceva™).

### BRIEF DESCRIPTION OF THE FIGURES

FIG. 1: Effect of drug treatments on animal weight after tumor implantation.

FIG. 2: Effect of drug treatments on tumor volume in LoVo human colon xenograft in nude mice.

FIG. 3: Representative treated tumors from efficacy study 540.

FIG. 4: Summary of toxicity for study 540.

FIG. 5: Summary of efficacy for study 540.

### DETAILED DESCRIPTION OF THE INVENTION

The term “cancer” in an animal refers to the presence of cells possessing characteristics typical of cancer-causing cells, such as uncontrolled proliferation, immortality, metastatic potential, rapid growth and proliferation rate, and certain characteristic morphological features. Often, cancer cells will be in the form of a tumor, but such cells may exist alone within an animal, or may circulate in the blood stream as independent cells, such as leukemic cells.

“Abnormal cell growth”, as used herein, unless otherwise indicated, refers to cell growth that is independent of normal regulatory mechanisms (e.g., loss of contact inhibition). This includes the abnormal growth of: (1) tumor cells (tumors) that proliferate by expressing a mutated tyrosine kinase or overexpression of a receptor tyrosine kinase; (2) benign and malignant cells of other proliferative diseases in which aberrant tyrosine kinase activation occurs; (4) any tumors that proliferate by receptor tyrosine kinases; (5) any tumors that proliferate by aberrant serine/threonine kinase activation; and (6) benign and malignant cells of other proliferative diseases in which aberrant serine/threonine kinase activation occurs.

The term “treating” as used herein, unless otherwise indicated, means reversing, alleviating, inhibiting the progress of, or preventing, either partially or completely, the growth of tumors, tumor metastases, or other cancer-causing or neoplastic cells in a patient. The term “treatment” as used herein, unless otherwise indicated, refers to the act of treating.

The phrase “a method of treating” or its equivalent, when applied to, for example, cancer refers to a procedure or course of action that is designed to reduce or eliminate the number of cancer cells in an animal, or to alleviate the symptoms of a cancer. “A method of treating” cancer or another proliferative disorder does not necessarily mean that the cancer cells or other disorder will, in fact, be eliminated, that the number of cells or disorder will, in fact, be reduced, or that the symptoms of a cancer or other disorder will, in fact, be alleviated. Often, a method of treating cancer will be performed even with a low likelihood of success, but which, given the medical history and estimated survival expectancy of an animal, is nevertheless deemed an overall beneficial course of action.

The term “therapeutically effective agent” means a composition that will elicit the biological or medical response of a tissue, system, animal or human that is being sought by the researcher, veterinarian, medical doctor or other clinician.

The term “therapeutically effective amount” or “effective amount” means the amount of the subject compound or combination that will elicit the biological or medical response of a tissue, system, animal or human that is being sought by the researcher, veterinarian, medical doctor or other clinician.

The data presented in the Examples herein below demonstrate that co-administration of oxaliplatin with an EGFR kinase inhibitor is effective for treatment of advanced cancers, such as colorectal cancer. Accordingly, the present invention provides a method for treating tumors or tumor metastases in a patient, comprising administering to the patient simultaneously or sequentially a therapeutically effective amount of an EGFR kinase inhibitor and oxaliplatin combination. In one embodiment the tumors or tumor metastases to be treated are colorectal tumors or tumor metastases.

The present invention further provides a method for treating tumors or tumor metastases in a patient, comprising administering to the patient simultaneously or sequentially a therapeutically effective amount of an EGFR kinase inhibitor and oxaliplatin combination, and in addition, one or more other cytotoxic, chemotherapeutic or anti-cancer agents, or compounds that enhance the effects of such agents.

In the context of this invention, additional other cytotoxic, chemotherapeutic or anti-cancer agents, or compounds that enhance the effects of such agents, include, for example: alkylating agents or agents with an alkylating action, such as cyclophosphamide (CTX; e.g. cytoxan®), chlorambucil (CHL; e.g. leukeran®), cisplatin (CisP; e.g. platinol®) busulfan (e.g. myleran®), melphalan, carmustine (BCNU), streptozotocin, triethylenemelamine (TEM), mitomycin C, and the like; anti-metabolites, such as methotrexate (MTX), etoposide (VP16; e.g. vepesid®), 6-mercaptopurine (6 MP), 6-thiocguanine (6TG), cytarabine (Ara-C), 5-fluorouracil (5-FU), capecitabine (e.g. Xeloda®), dacarbazine (DTIC), and the like; antibiotics, such as actinomycin D, doxorubicin (DXR; e.g. adriamycin®), daunorubicin (daunomycin), bleomycin, mithramycin and the like; alkaloids, such as vinca alkaloids such as vincristine (VCR), vinblastine, and the like; and other antitumor agents, such as paclitaxel (e.g. taxol®) and pactitaxel derivatives, the cytostatic agents, glucocorticoids such as dexamethasone (DEX; e.g. decadron®) and corticosteroids such as prednisone, nucleoside enzyme inhibitors such as hydroxyurea, amino acid depleting enzymes such as asparaginase, leucovorin, folinic acid, raltitrexed, and other folic acid derivatives, and similar, diverse antitumor agents. The following agents may also be used as additional agents: amifostine (e.g. ethyol®), dactinomycin, mechlorethamine (nitrogen mustard), streptozocin, cyclophosphamide, lomustine (CCNU), doxorubicin lipo (e.g. doxil®), gemcitabine (e.g. gemzar®), daunorubicin lipo (e.g. daunoxome®), procarbazine, mitomycin, docetaxel (e.g. taxotere®), aldesleukin, carboplatin, cladribine, camptothecin, CPT 11 (irinotecan), 10-hydroxy 7-ethyl-camptothecin (SN38), floxuridine, fludarabine, ifosfamide, idarubicin, mesna, interferon alpha, interferon beta, mitoxantrone, topotecan, leuprolide, megestrol, melphalan, mercaptopurine, plicamycin, mitotane, pegaspargase, pentostatin, pipobroman, plicamycin, tamoxifen, teniposide, testolactone, thioguanine, thiotepa, uracil mustard, vinorelbine, chlorambucil.

The present invention further provides a method for treating tumors or tumor metastases in a patient, comprising administering to the patient simultaneously or sequentially a therapeutically effective amount of an EGFR kinase inhibitor and oxaliplatin combination, and in addition, one or more anti-hormonal agents. As used herein, the term “anti-hormonal agent” includes natural or synthetic organic or peptidic compounds that act to regulate or inhibit hormone action on tumors.

Antihormonal agents include, for example: steroid receptor antagonists, anti-estrogens such as tamoxifen, raloxifene, aromatase inhibiting 4(5)-imidazoles, other aromatase inhibitors, 42-hydroxytamoxifen, trioxifene, keoxifene, LY 117018, onapristone, and toremifene (e.g. Fareston®); anti-androgens such as flutamide, nilutamide, bicalutamide, leuprolide, and goserelin; and pharmaceutically acceptable salts, acids or derivatives of any of the above; agonists and/or antagonists of glycoprotein hormones such as follicle stimulating hormone (FSH), thyroid stimulating hormone (TSH), and luteinizing hormone (LH) and LHRH (leuteinizing hormone-releasing hormone); the LHRH agonist goserelin acetate, commercially available as Zoladex® (AstraZeneca); the LHRH antagonist D-alaninamide N-acetyl-3-(2-naphthalenyl)-D-alanyl-4-chloro-D-phenylalanyl-3-(3-pyridinyl)-D-alanyl-L-seryl-N-6-(3-pyridinylcarbonyl)-L-lysyl-N-6-(3-pyridinylcarbonyl)-D-lysyl-L-leucyl-N6-(1-methylethyl)-L-lysyl-L-proline (e.g Antide®, Ares-Serono); the LHRH antagonist ganirelix acetate; the steroidal anti-androgens cyproterone acetate (CPA) and megestrol acetate, commercially available as Megace® (Bristol-Myers Oncology); the nonsteroidal anti-androgen flutamide (2-methyl-N-[4,20-nitro-3-(trifluoromethyl) phenylpropanamide), commercially available as Eulexin® (Schering Corp.); the non-steroidal anti-androgen nilutamide, (5,5-dimethyl-3-[4-nitro-3-(trifluoromethyl-4′-nitrophenyl)-4,4-dimethyl-imidazolidine-dione); and antagonists for other non-permissive receptors, such as antagonists for RAR, RXR, TR, VDR, and the like.

The use of the cytotoxic and other anticancer agents described above in chemotherapeutic regimens is generally well characterized in the cancer therapy arts, and their use herein falls under the same considerations for monitoring tolerance and effectiveness and for controlling administration routes and dosages, with some adjustments. For example, the actual dosages of the cytotoxic agents may vary depending upon the patient's cultured cell response determined by using histoculture methods. Generally, the dosage will be reduced compared to the amount used in the absence of additional other agents.

Typical dosages of an effective cytotoxic agent can be in the ranges recommended by the manufacturer, and where indicated by in vitro responses or responses in animal models, can be reduced by up to about one order of magnitude concentration or amount. Thus, the actual dosage will depend upon the judgment of the physician, the condition of the patient, and the effectiveness of the therapeutic method based on the in vitro responsiveness of the primary cultured malignant cells or histocultured tissue sample, or the responses observed in the appropriate animal models.

In the context of this invention, of the above additional other cytotoxic, chemotherapeutic or anticancer agents the compound 5-fluorouracil is preferred. Conveniently, a combination of 5-fluorouracil and leucovoran can be used with the EGFR kinase inhibitor and oxaliplatin combination of this invention. A 5-fluorouracil, leucovoran and oxaliplatin combination is often referred to as FOLFOX4.

The present invention further provides a method for treating tumors or tumor metastases in a patient, comprising administering to the patient simultaneously or sequentially a therapeutically effective amount of an EGFR kinase inhibitor and oxaliplatin combination, and in addition one or more angiogenesis inhibitors.

Anti-angiogenic agents include, for example: VEGFR inhibitors, such as SU-5416 and SU-6668 (Sugen Inc. of South San Francisco, Calif., USA), or as described in, for example International Application Nos. WO 99/24440, WO 99/62890, WO 95/21613, WO 99/61422, WO 98/50356, WO 99/10349, WO 97/32856, WO 97/22596, WO 98/54093, WO 98/02438, WO 99/16755, and WO 98/02437, and U.S. Pat. Nos. 5,883,113, 5,886,020, 5,792,783, 5,834,504 and 6,235,764; VEGF inhibitors such as IM862 (Cytran Inc. of Kirkland, Wash., USA); angiozyme, a synthetic ribozyme from Ribozyme (Boulder, Colo.) and Chiron (Emeryville, Calif.); and antibodies to VEGF, such as bevacizumab (e.g. Avastin™, Genentech, South San Francisco, Calif.), a recombinant humanized antibody to VEGF; integrin receptor antagonists and integrin antagonists, such as αᵥβ₃, αᵥβ₅ and αᵥβ₆ integrins, and subtypes thereof, e.g. cilengitide (EMD 121974), or the anti-integrin antibodies, such as for example αᵥβ₃ specific humanized antibodies (e.g. Vitaxin®); factors such as IFN-alpha (U.S. Pat. Nos. 41,530,901, 4,503,035, and 5,231,176); angiostatin and plasminogen fragments (e.g. kringle 1-4, kringle 5, kringle 1-3 (O'Reilly, M. S. et al. (1994) Cell 79:315-328; Cao et al. (1996) J. Biol. Chem. 271: 29461-29467; Cao et al. (1997) J. Biol. Chem. 272:22924-22928); endostatin (O'Reilly, M. S. et al. (1997) Cell 88:277; and International Patent Publication No. WO 97/15666); thrombospondin (TSP-1; Frazier, (1991) Curr. Opin. Cell Biol. 3:792); platelet factor 4 (PF4); plasminogen activator/urokinase inhibitors; urokinase receptor antagonists; heparinases; fumagillin analogs such as TNP-4701; suramin and suramin analogs; angiostatic steroids; bFGF antagonists; flk-1 and flt-1 antagonists; anti-angiogenesis agents such as MMP-2 (matrix-metalloproteinase 2) inhibitors and MMP-9 (matrix-metalloproteinase 9) inhibitors. Examples of useful matrix metalloproteinase inhibitors are described in International Patent Publication Nos. WO 96/33172, WO 96/27583, WO 98/07697, WO 98/03516, WO 98/34918, WO 98/34915, WO 98/33768, WO 98/30566, WO 90/05719, WO 99/52910, WO 99/52889, WO 99/29667, and WO 99/07675, European Patent Publication Nos. 818,442, 780,386, 1,004,578, 606,046, and 931,788; Great Britain Patent Publication No. 9912961, and U.S. Pat. Nos. 5,863,949 and 5,861,510. Preferred MMP-2 and MMP-9 inhibitors are those that have little or no activity inhibiting MMP-1. More preferred, are those that selectively inhibit MMP-2 and/or MMP-9 relative to the other matrix-metalloproteinases (i.e. MMP-1, MMP-3, MMP-4, MMP-5, MMP-6, MMP-7, MMP-8, MMP-10, MMP-11, MMP-12, and MMP-13).

The present invention further provides a method for treating tumors or tumor metastases in a patient, comprising administering to the patient simultaneously or sequentially a therapeutically effective amount of an EGFR kinase inhibitor and oxaliplatin combination, and in addition one or more tumor cell pro-apoptotic or apoptosis-stimulating agents.

The present invention further provides a method for treating tumors or tumor metastases in a patient, comprising administering to the patient simultaneously or sequentially a therapeutically effective amount of an EGFR kinase inhibitor and oxaliplatin combination, and in addition one or more signal transduction inhibitors.

Signal transduction inhibitors include, for example: erbB2 receptor inhibitors, such as organic molecules, or antibodies that bind to the erbB2 receptor, for example, trastuzumab (e.g. Herceptin®); inhibitors of other protein tyrosine-kinases, e.g. imitinib (e.g. Gleevec®); ras inhibitors; raf inhibitors; MEK inhibitors; mTOR inhibitors; cyclin dependent kinase inhibitors; protein kinase C inhibitors; and PDK-1 inhibitors (see Dancey, J. and Sausville, E. A. (2003) Nature Rev. Drug Discovery 2:92-313, for a description of several examples of such inhibitors, and their use in clinical trials for the treatment of cancer).

ErbB2 receptor inhibitors include, for example: ErbB2 receptor inhibitors, such as GW-282974 (Glaxo Wellcome plc), monoclonal antibodies such as AR-209 (Aronex Pharmaceuticals Inc. of The Woodlands, Tex., USA) and 2B-1 (Chiron), and erbB2 inhibitors such as those described in International Publication Nos. WO 98/02434, WO 99/35146, WO 99/35132, WO 98/02437, WO 97/13760, and WO 95/19970, and U.S. Pat. Nos. 5,587,458, 5,877,305, 6,465,449 and 6,541,481.

The present invention further thus provides a method for treating tumors or tumor metastases in a patient, comprising administering to the patient simultaneously or sequentially a therapeutically effective amount of an EGFR kinase inhibitor and oxaliplatin combination, and in addition an anti-HER2 antibody or an immunotherapeutically active fragment thereof.

The present invention further provides a method for treating tumors or tumor metastases in a patient, comprising administering to the patient simultaneously or sequentially a therapeutically effective amount of an EGFR kinase inhibitor and oxaliplatin combination, and in addition one or more additional anti-proliferative agents.

Additional antiproliferative agents include, for example: Inhibitors of the enzyme farnesyl protein transferase and inhibitors of the receptor tyrosine kinase PDGFR, including the compounds disclosed and claimed in U.S. Pat. Nos. 6,080,769, 6,194,438, 6,258,824, 6,586,447, 6,071,935, 6,495,564, 6,150,377, 6,596,735 and 6,479,513, and International Patent Publication WO 01/40217.

The present invention further provides a method for treating tumors or tumor metastases in a patient, comprising administering to the patient simultaneously or sequentially a therapeutically effective amount of an EGFR kinase inhibitor and oxaliplatin combination, and in addition a COX II (cyclooxygenase II) inhibitor. Examples of useful COX-II inhibitors include alecoxib (e.g. Celebrex™), valdecoxib, and rofecoxib.

The present invention further provides a method for treating tumors or tumor metastases in a patient, comprising administering to the patient simultaneously or sequentially a therapeutically effective amount of an EGFR kinase inhibitor and oxaliplatin combination, and in addition treatment with radiation or a radiopharmaceutical.

The source of radiation can be either external or internal to the patient being treated. When the source is external to the patient, the therapy is known as external beam radiation therapy (EBRT). When the source of radiation is internal to the patient, the treatment is called brachytherapy (BT). Radioactive atoms for use in the context of this invention can be selected from the group including, but not limited to, radium, cesium-137, iridium-192, americium-241, gold-198, cobalt-57, copper-67, technetium-99, iodine-123, iodine-131, and indium-111. Where the EGFR kinase inhibitor according to this invention is an antibody, it is also possible to label the antibody with such radioactive isotopes.

Radiation therapy is a standard treatment for controlling unresectable or inoperable tumors and/or tumor metastases. Improved results have been seen when radiation therapy has been combined with chemotherapy. Radiation therapy is based on the principle that high-dose radiation delivered to a target area will result in the death of reproductive cells in both tumor and normal tissues. The radiation dosage regimen is generally defined in terms of radiation absorbed dose (Gy), time and fractionation, and must be carefully defined by the oncologist. The amount of radiation a patient receives will depend on various considerations, but the two most important are the location of the tumor in relation to other critical structures or organs of the body, and the extent to which the tumor has spread. A typical course of treatment for a patient undergoing radiation therapy will be a treatment schedule over a 1 to 6 week period, with a total dose of between 10 and 80 Gy administered to the patient in a single daily fraction of about 1.8 to 2.0 Gy, 5 days a week. In a preferred embodiment of this invention there is synergy when tumors in human patients are treated with the combination treatment of the invention and radiation. In other words, the inhibition of tumor growth by means of the agents comprising the combination of the invention is enhanced when combined with radiation, optionally with additional chemotherapeutic or anticancer agents. Parameters of adjuvant radiation therapies are, for example, contained in International Patent Publication WO 99/60023.

The present invention further provides a method for treating tumors or tumor metastases in a patient, comprising administering to the patient simultaneously or sequentially a therapeutically effective amount of an EGFR kinase inhibitor and oxaliplatin combination, and in addition treatment with one or more agents capable of enhancing antitumor immune responses.

Agents capable of enhancing antitumor immune responses include, for example: CTLA4 (cytotoxic lymphocyte antigen 4) antibodies (e.g. MDX-CTLA4), and other agents capable of blocking CTLA4. Specific CTLA4 antibodies that can be used in the present invention include those described in U.S. Pat. No. 6,682,736.

The present invention further provides a method for reducing the side effects caused by the treatment of tumors or tumor metastases in a patient with an EGFR kinase inhibitor or oxaliplatin, comprising administering to the patient simultaneously or sequentially a therapeutically effective amount of an EGFR kinase inhibitor and oxaliplatin combination, in amounts that are effective to produce an additive, or a superadditive or synergistic antitumor effect, and that are effective at inhibiting the growth of the tumor.

The present invention further provides a method for the treatment of cancer, comprising administering to a subject in need of such treatment (i) an effective first amount of an EGFR kinase inhibitor, or a pharmaceutically acceptable salt thereof; and (ii) an effective second amount of oxaliplatin.

The present invention also provides a method for the treatment of cancer, comprising administering to a subject in need of such treatment (i) a sub-therapeutic first amount of the EGFR kinase inhibitor erlotinib, or a pharmaceutically acceptable salt thereof; and (ii) a sub-therapeutic second amount of oxaliplatin.

Additionally, the present invention provides a pharmaceutical composition comprising an EGFR inhibitor and oxaliplatin in a pharmaceutically acceptable carrier.

As used herein, the term “patient” preferably refers to a human in need of treatment with an EGFR kinase inhibitor for any purpose, and more preferably a human in need of such a treatment to treat cancer, or a precancerous condition or lesion. However, the term “patient” can also refer to non-human animals, preferably mammals such as dogs, cats, horses, cows, pigs, sheep and non-human primates, among others, that are in need of treatment with an EGFR kinase inhibitor.

In a preferred embodiment, the patient is a human in need of treatment for cancer, or a precancerous condition or lesion. The cancer is preferably any cancer treatable, either partially or completely, by administration of an EGFR kinase inhibitor. The cancer may be, for example, lung cancer, non small cell lung (NSCL) cancer, bronchioloalviolar cell lung cancer, bone cancer, pancreatic cancer, skin cancer, cancer of the head or neck, cutaneous or intraocular melanoma, uterine cancer, ovarian cancer, rectal cancer, cancer of the anal region, stomach cancer, gastric cancer, colon cancer, breast cancer, uterine cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, cancer of the bladder, cancer of the kidney or ureter, renal cell carcinoma, carcinoma of the renal pelvis, mesothelioma, hepatocellular cancer, biliary cancer, chronic or acute leukemia, lymphocytic lymphomas, neoplasms of the central nervous system (CNS), spinal axis tumors, brain stem glioma, glioblastoma multiforme, astrocytomas, schwannomas, ependymomas, medulloblastomas, meningiomas, squamous cell carcinomas, pituitary adenomas, including refractory versions of any of the above cancers, or a combination of one or more of the above cancers. The precancerous condition or lesion includes, for example, the group consisting of oral leukoplakia, actinic keratosis (solar keratosis), precancerous polyps of the colon or rectum, gastric epithelial dysplasia, adenomatous dysplasia, hereditary nonpolyposis colon cancer syndrome (HNPCC), Barrett's esophagus, bladder dysplasia, and precancerous cervical conditions.

For purposes of the present invention, “co-administration of” and “co-administering” oxaliplatin with an EGFR kinase inhibitor (both components referred to hereinafter as the “two active agents”) refer to any administration of the two active agents, either separately or together, where the two active agents are administered as part of an appropriate dose regimen designed to obtain the benefit of the combination therapy. Thus, the two active agents can be administered either as part of the same pharmaceutical composition or in separate pharmaceutical compositions. Oxaliplatin can be administered prior to, at the same time as, or subsequent to administration of the EGFR kinase inhibitor, or in some combination thereof. Where the EGFR kinase inhibitor is administered to the patient at repeated intervals, e.g., during a standard course of treatment, oxaliplatin can be administered prior to, at the same time as, or subsequent to, each administration of the EGFR kinase inhibitor, or some combination thereof, or at different intervals in relation to the EGFR kinase inhibitor treatment, or in a single dose prior to, at any time during, or subsequent to the course of treatment with the EGFR kinase inhibitor.

The EGFR kinase inhibitor will typically be administered to the patient in a dose regimen that provides for the most effective treatment of the cancer (from both efficacy and safety perspectives) for which the patient is being treated, as known in the art, and as disclosed, e.g. in International Patent Publication No. WO 01/34574. In conducting the treatment method of the present invention, the EGFR kinase inhibitor can be administered in any effective manner known in the art, such as by oral, topical, intravenous, intra-peritoneal, intramuscular, intra-articular, subcutaneous, intranasal, intra-ocular, vaginal, rectal, or intradermal routes, depending upon the type of cancer being treated, the type of EGFR kinase inhibitor being used (e.g., small molecule, antibody, RNAi or antisense construct), and the medical judgement of the prescribing physician as based, e.g., on the results of published clinical studies.

The amount of EGFR kinase inhibitor administered and the timing of EGFR kinase inhibitor administration will depend on the type (species, gender, age, weight, etc.) and condition of the patient being treated, the severity of the disease or condition being treated, and on the route of administration. For example, small molecule EGFR kinase inhibitors can be administered to a patient in doses ranging from 0.001 to 100 mg/kg of body weight per day or per week in single or divided doses, or by continuous infusion (see for example, International Patent Publication No. WO 01/34574). In particular, erlotinib HCl can be administered to a patient in doses ranging from 5-200 mg per day, or 100-1600 mg per week, in single or divided doses, or by continuous infusion. A preferred dose is 150 mg/day. Antibody-based EGFR kinase inhibitors, or antisense, RNAi or ribozyme constructs, can be administered to a patient in doses ranging from 0.1 to 100 mg/kg of body weight per day or per week in single or divided doses, or by continuous infusion. In some instances, dosage levels below the lower limit of the aforesaid range may be more than adequate, while in other cases still larger doses may be employed without causing any harmful side effect, provided that such larger doses are first divided into several small doses for administration throughout the day.

The EGFR kinase inhibitors and oxaliplatin can be administered either separately or together by the same or different routes, and in a wide variety of different dosage forms. For example, the EGFR kinase inhibitor is preferably administered orally or parenterally, whereas oxaliplatin is preferably administered parenterally. Where the EGFR kinase inhibitor is erlotinib HCl (Tarceva™), oral administration is preferable.

The EGFR kinase inhibitor can be administered with various pharmaceutically acceptable inert carriers in the form of tablets, capsules, lozenges, troches, hard candies, powders, sprays, creams, salves, suppositories, jellies, gels, pastes, lotions, ointments, elixirs, syrups, and the like. Administration of such dosage forms can be carried out in single or multiple doses. Carriers include solid diluents or fillers, sterile aqueous media and various non-toxic organic solvents, etc. Oral pharmaceutical compositions can be suitably sweetened and/or flavored.

The EGFR kinase inhibitor and oxaliplatin can be combined together with various pharmaceutically acceptable inert carriers in the form of sprays, creams, salves, suppositories, jellies, gels, pastes, lotions, ointments, and the like. Administration of such dosage forms can be carried out in single or multiple doses. Carriers include solid diluents or fillers, sterile aqueous media, and various non-toxic organic solvents, etc.

All formulations comprising proteinaceous EGFR kinase inhibitors should be selected so as to avoid denaturation and/or degradation and loss of biological activity of the inhibitor.

Methods of preparing pharmaceutical compositions comprising an EGFR kinase inhibitor are known in the art, and are described, e.g. in International Patent Publication No. WO 01/34574. Methods of preparing pharmaceutical compositions comprising oxaliplatin are also well known in the art. In view of the teaching of the present invention, methods of preparing pharmaceutical compositions comprising both an EGFR kinase inhibitor and oxaliplatin will be apparent from the above-cited publications and from other known references, such as Remington's Pharmaceutical Sciences, Mack Publishing Company, Easton, Pa., 18^{th} edition (1990).

For oral administration of EGFR kinase inhibitors, tablets containing one or both of the active agents are combined with any of various excipients such as, for example, micro-crystalline cellulose, sodium citrate, calcium carbonate, dicalcium phosphate and glycine, along with various disintegrants such as starch (and preferably corn, potato or tapioca starch), alginic acid and certain complex silicates, together with granulation binders like polyvinyl pyrrolidone, sucrose, gelatin and acacia. Additionally, lubricating agents such as magnesium stearate, sodium lauryl sulfate and talc are often very useful for tableting purposes. Solid compositions of a similar type may also be employed as fillers in gelatin capsules; preferred materials in this connection also include lactose or milk sugar as well as high molecular weight polyethylene glycols. When aqueous suspensions and/or elixirs are desired for oral administration, the EGFR kinase inhibitor may be combined with various sweetening or flavoring agents, coloring matter or dyes, and, if so desired, emulsifying and/or suspending agents as well, together with such diluents as water, ethanol, propylene glycol, glycerin and various like combinations thereof.

For parenteral administration of either or both of the active agents, solutions in either sesame or peanut oil or in aqueous propylene glycol may be employed, as well as sterile aqueous solutions comprising the active agent or a corresponding water-soluble salt thereof. Such sterile aqueous solutions are preferably suitably buffered, and are also preferably rendered isotonic, e.g., with sufficient saline or glucose. These particular aqueous solutions are especially suitable for intravenous, intramuscular, subcutaneous and intraperitoneal injection purposes. The oily solutions are suitable for intra-articular, intramuscular and subcutaneous injection purposes. The preparation of all these solutions under sterile conditions is readily accomplished by standard pharmaceutical techniques well known to those skilled in the art. Any parenteral formulation selected for administration of proteinaceous EGFR kinase inhibitors should be selected so as to avoid denaturation and loss of biological activity of the inhibitor.

Additionally, it is possible to topically administer either or both of the active agents, by way of, for example, creams, lotions, jellies, gels, pastes, ointments, salves and the like, in accordance with standard pharmaceutical practice. For example, a topical formulation comprising either an EGFR kinase inhibitor or oxaliplatin in about 0.1% (w/v) to about 5% (w/v) concentration can be prepared.

For veterinary purposes, the active agents can be administered separately or together to animals using any of the forms and by any of the routes described above. In a preferred embodiment, the EGFR kinase inhibitor is administered in the form of a capsule, bolus, tablet, liquid drench, by injection or as an implant. As an alternative, the EGFR kinase inhibitor can be administered with the animal feedstuff, and for this purpose a concentrated feed additive or premix may be prepared for a normal animal feed. The oxaliplatin is preferably administered in the form of liquid drench, by injection or as an implant. Such formulations are prepared in a conventional manner in accordance with standard veterinary practice.

The present invention further provides a kit comprising a single container comprising both an EGFR kinase inhibitor and oxaliplatin. The present invention further provides a kit comprising a first container comprising an EGFR kinase inhibitor and a second container comprising oxaliplatin. In a preferred embodiment, the kit containers may further include a pharmaceutically acceptable carrier. The kit may further include a sterile diluent, which is preferably stored in a separate additional container. The kit may further include a package insert comprising printed instructions directing the use of the combined treatment as a method for treating cancer.

As used herein, the term “EGFR kinase inhibitor” refers to any EGFR kinase inhibitor that is currently known in the art or that will be identified in the future, and includes any chemical entity that, upon administration to a patient, results in inhibition of a biological activity associated with activation of the EGF receptor in the patient, including any of the downstream biological effects otherwise resulting from the binding to EGFR of its natural ligand. Such EGFR kinase inhibitors include any agent that can block EGFR activation or any of the downstream biological effects of EGFR activation that are relevant to treating cancer in a patient. Such an inhibitor can act by binding directly to the intracellular domain of the receptor and inhibiting its kinase activity. Alternatively, such an inhibitor can act by occupying the ligand binding site or a portion thereof of the EGFR receptor, thereby making the receptor inaccessible to its natural ligand so that its normal biological activity is prevented or reduced. Alternatively, such an inhibitor can act by modulating the dimerization of EGFR polypeptides, or interaction of EGFR polypeptide with other proteins, or enhance ubiquitination and endocytotic degradation of EGFR. EGFR kinase inhibitors include but are not limited to low molecular weight inhibitors, antibodies or antibody fragments, antisense constructs, small inhibitory RNAs (i.e. RNA interference by dsRNA; RNAi), and ribozymes. In a preferred embodiment, the EGFR kinase inhibitor is a small organic molecule or an antibody that binds specifically to the human EGFR.

EGFR kinase inhibitors that include, for example quinazoline EGFR kinase inhibitors, pyrido-pyrimidine EGFR kinase inhibitors, pyrimido-pyrimidine EGFR kinase inhibitors, pyrrolo-pyrimidine EGFR kinase inhibitors, pyrazolo-pyrimidine EGFR kinase inhibitors, phenylamino-pyrimidine EGFR kinase inhibitors, oxindole EGFR kinase inhibitors, indolocarbazole EGFR kinase inhibitors, phthalazine EGFR kinase inhibitors, isoflavone EGFR kinase inhibitors, quinalone EGFR kinase inhibitors, and tyrphostin EGFR kinase inhibitors, such as those described in the following patent publications, and all pharmaceutically acceptable salts and solvates of said EGFR kinase inhibitors: International Patent Publication Nos. WO 96/33980, WO 96/30347, WO 97/30034, WO 97/30044, WO 97/38994, WO 97/49688, WO 98/02434, WO 97/38983, WO 95/19774, WO 95/19970, WO 97/13771, WO 98/02437, WO 98/02438, WO 97/32881, WO 98/33798, WO 97/32880, WO 97/3288, WO 97/02266, WO 97/27199, WO 98/07726, WO 97/34895, WO 96/31510, WO 98/14449, WO 98/14450, WO 98/14451, WO 95/09847, WO 97/19065, WO 98/17662, WO 99/35146, WO 99/35132, WO 99/07701, and WO 92/20642; European Patent Application Nos. EP 520722, EP 566226, EP 787772, EP 837063, and EP 682027; U.S. Pat. Nos. 5,747,498, 5,789,427, 5,650,415, and 5,656,643; and German Patent Application No. DE 19629652. Additional non-limiting examples of low molecular weight EGFR kinase inhibitors include any of the EGFR kinase inhibitors described in Traxler, P., 1998, Exp. Opin. Ther. Patents 8(12):1599-1625.

Specific preferred examples of low molecular weight EGFR kinase inhibitors that can be used according to the present invention include [6,7-bis(2-methoxyethoxy)-4-quinazolin-4-yl]-(3-ethynylphenyl) amine (also known as OSI-774, erlotinib, or Tarceva™ (erlotinib HCl); OSI Pharmaceuticals/Genentech/Roche) (U.S. Pat. No. 5,747,498; International Patent Publication No. WO 01/34574, and Moyer, J. D. et al. (1997) Cancer Res. 57:4838-4848); C₁₋₁₀₃₃ (formerly known as PD183805; Pfizer) (Sherwood et al., 1999, Proc. Am. Assoc. Cancer Res. 40:723); PD-158780 (Pfizer); AG-1478 (University of California); CGP-59326 (Novartis); PKI-166 (Novartis); EKB-569 (Wyeth); GW-2016 (also known as GW-572016 or lapatinib ditosylate; GSK); and gefitinib (also known as ZD1839 or Iressa™; Astrazeneca) (Woodburn et al., 1997, Proc. Am. Assoc. Cancer Res. 38:633). A particularly preferred low molecular weight EGFR kinase inhibitor that can be used according to the present invention is [6,7-bis(2-methoxyethoxy)-4-quinazolin-4-yl]-(3-ethynylphenyl) amine (i.e. erlotinib), its hydrochloride salt (i.e. erlotinib HCl, Tarceva™), or other salt forms (e.g. erlotinib mesylate).

Antibody-based EGFR kinase inhibitors include any anti-EGFR antibody or antibody fragment that can partially or completely block EGFR activation by its natural ligand. Non-limiting examples of antibody-based EGFR kinase inhibitors include those described in Modjtahedi, H., et al., 1993, Br. J. Cancer 67:247-253; Teramoto, T., et al., 1996, Cancer 77:639-645; Goldstein et al., 1995, Clin. Cancer Res. 1:1311-1318; Huang, S. M., et al., 1999, Cancer Res. 15:59(8):1935-40; and Yang, X., et al., 1999, Cancer Res. 59:1236-1243. Thus, the EGFR kinase inhibitor can be monoclonal antibody Mab E7.6.3 (Yang, X. D. et al. (1999) Cancer Res. 59:1236-43), or Mab C225 (ATCC Accession No. HB-8508), or an antibody or antibody fragment having the binding specificity thereof. Suitable monoclonal antibody EGFR kinase inhibitors include, but are not limited to, IMC-C225 (also known as cetuximab or Erbitux™; mclone Systems), ABX-EGF (Abgenix), EMD 72000 (Merck KgaA, Darmstadt), RH3 (York Medical Bioscience Inc.), and MDX-447 (Medarex/Merck KgaA).

Additional antibody-based EGFR kinase inhibitors can be raised according to known methods by administering the appropriate antigen or epitope to a host animal selected, e.g., from pigs, cows, horses, rabbits, goats, sheep, and mice, among others. Various adjuvants known in the art can be used to enhance antibody production.

Although antibodies useful in practicing the invention can be polyclonal, monoclonal antibodies are preferred. Monoclonal antibodies against EGFR can be prepared and isolated using any technique that provides for the production of antibody molecules by continuous cell lines in culture. Techniques for production and isolation include but are not limited to the hybridoma technique originally described by Kohler and Milstein (Nature, 1975, 256: 495-497); the human B-cell hybridoma technique (Kosbor et al., 1983, Immunology Today 4:72; Cote et al., 1983, Proc. Natl. Acad. Sci. USA 80: 2026-2030); and the EBV-hybridoma technique (Cole et al, 1985, Monoclonal Antibodies and Cancer Therapy, Alan R. Liss, Inc., pp. 77-96).

Alternatively, techniques described for the production of single chain antibodies (see, e.g., U.S. Pat. No. 4,946,778) can be adapted to produce anti-EGFR single chain antibodies. Antibody-based EGFR kinase inhibitors useful in practicing the present invention also include anti-EGFR antibody fragments including but not limited to F(ab′).sub.2 fragments, which can be generated by pepsin digestion of an intact antibody molecule, and Fab fragments, which can be generated by reducing the disulfide bridges of the F(ab′).sub.2 fragments. Alternatively, Fab and/or scFv expression libraries can be constructed (see, e.g., Huse et al., 1989, Science 246: 1275-1281) to allow rapid identification of fragments having the desired specificity to EGFR.

Techniques for the production and isolation of monoclonal antibodies and antibody fragments are well-known in the art, and are described in Harlow and Lane, 1988, Antibodies: A Laboratory Manual, Cold Spring Harbor Laboratory, and in J. W. Goding, 1986, Monoclonal Antibodies: Principles and Practice, Academic Press, London. Humanized anti-EGFR antibodies and antibody fragments can also be prepared according to known techniques such as those described in Vaughn, T. J. et al., 1998, Nature Biotech. 16:535-539 and references cited therein, and such antibodies or fragments thereof are also useful in practicing the present invention.

EGFR kinase inhibitors for use in the present invention can alternatively be based on antisense oligonucleotide constructs. Anti-sense oligonucleotides, including anti-sense RNA molecules and anti-sense DNA molecules, would act to directly block the translation of EGFR mRNA by binding thereto and thus preventing protein translation or increasing mRNA degradation, thus decreasing the level of EGFR kinase protein, and thus activity, in a cell. For example, antisense oligonucleotides of at least about 15 bases and complementary to unique regions of the mRNA transcript sequence encoding EGFR can be synthesized, e.g., by conventional phosphodiester techniques and administered by e.g., intravenous injection or infusion. Methods for using antisense techniques for specifically inhibiting gene expression of genes whose sequence is known are well known in the art (e.g. see U.S. Pat. Nos. 6,566,135; 6,566,131; 6,365,354; 6,410,323; 6,107,091; 6,046,321; and 5,981,732).

Small inhibitory RNAs (siRNAs) can also function as EGFR kinase inhibitors for use in the present invention. EGFR gene expression can be reduced by contacting the tumor, subject or cell with a small double stranded RNA (dsRNA), or a vector or construct causing the production of a small double stranded RNA, such that expression of EGFR is specifically inhibited (i.e. RNA interference or RNAi). Methods for selecting an appropriate dsRNA or dsRNA-encoding vector are well known in the art for genes whose sequence is known (e.g. see Tuschi, T., et al. (1999) Genes Dev. 13(24):3191-3197; Elbashir, S. M. et al. (2001) Nature 411:494-498; Hannon, G. J. (2002) Nature 418:244-251; McManus, M. T. and Sharp, P. A. (2002) Nature Reviews Genetics 3:737-747; Bremmelkamp, T. R. et al. (2002) Science 296:550-553; U.S. Pat. Nos. 6,573,099 and 6,506,559; and International Patent Publication Nos. WO 01/36646, WO 99/32619, and WO 01/68836).

Ribozymes can also function as EGFR kinase inhibitors for use in the present invention. Ribozymes are enzymatic RNA molecules capable of catalyzing the specific cleavage of RNA. The mechanism of ribozyme action involves sequence specific hybridization of the ribozyme molecule to complementary target RNA, followed by endonucleolytic cleavage. Engineered hammerhead motif ribozyme molecules that specifically and efficiently catalyze endonucleolytic cleavage of EGFR mRNA sequences are thereby useful within the scope of the present invention. Specific ribozyme cleavage sites within any potential RNA target are initially identified by scanning the target molecule for ribozyme cleavage sites, which typically include the following sequences, GUA, GUU, and GUC. Once identified, short RNA sequences of between about 15 and 20 ribonucleotides corresponding to the region of the target gene containing the cleavage site can be evaluated for predicted structural features, such as secondary structure, that can render the oligonucleotide sequence unsuitable. The suitability of candidate targets can also be evaluated by testing their accessibility to hybridization with complementary oligonucleotides, using, e.g., ribonuclease protection assays.

Both antisense oligonucleotides and ribozymes useful as EGFR kinase inhibitors can be prepared by known methods. These include techniques for chemical synthesis such as, e.g., by solid phase phosphoramadite chemical synthesis. Alternatively, anti-sense RNA molecules can be generated by in vitro or in vivo transcription of DNA sequences encoding the RNA molecule. Such DNA sequences can be incorporated into a wide variety of vectors that incorporate suitable RNA polymerase promoters such as the T7 or SP6 polymerase promoters. Various modifications to the oligonucleotides of the invention can be introduced as a means of increasing intracellular stability and half-life. Possible modifications include but are not limited to the addition of flanking sequences of ribonucleotides or deoxyribonucleotides to the 5′ and/or 3′ ends of the molecule, or the use of phosphorothioate or 2′-O-methyl rather than phosphodiesterase linkages within the oligonucleotide backbone.

The invention also encompasses a pharmaceutical composition that is comprised of an EGFR kinase inhibitor and oxaliplatin combination in combination with a pharmaceutically acceptable carrier.

Preferably the composition is comprised of a pharmaceutically acceptable carrier and a non-toxic therapeutically effective amount of an EGFR kinase inhibitor compound and oxaliplatin combination (including pharmaceutically acceptable salts of each component thereof).

Moreover, within this preferred embodiment, the invention encompasses a pharmaceutical composition for the treatment of disease, the use of which results in the inhibition of growth of neoplastic cells, benign or malignant tumors, or metastases, comprising a pharmaceutically acceptable carrier and a non-toxic therapeutically effective amount of an EGFR kinase inhibitor compound and oxaliplatin combination (including pharmaceutically acceptable salts of each component thereof).

The term “pharmaceutically acceptable salts” refers to salts prepared from pharmaceutically acceptable non-toxic bases or acids. When a compound of the present invention is acidic, its corresponding salt can be conveniently prepared from pharmaceutically acceptable non-toxic bases, including inorganic bases and organic bases. Salts derived from such inorganic bases include aluminum, ammonium, calcium, copper (cupric and cuprous), ferric, ferrous, lithium, magnesium, manganese (manganic and manganous), potassium, sodium, zinc and the like salts. Particularly preferred are the ammonium, calcium, magnesium, potassium and sodium slats. Salts derived from pharmaceutically acceptable organic non-toxic bases include salts of primary, secondary, and tertiary amines, as well as cyclic amines and substituted amines such as naturally occurring and synthesized substituted amines. Other pharmaceutically acceptable organic non-toxic bases from which salts can be formed include ion exchange resins such as, for example, arginine, betaine, caffeine, choline, N′,N′-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylameine, trimethylamine, tripropylamine, tromethamine and the like.

When a compound of the present invention is basic, its corresponding salt can be conveniently prepared from pharmaceutically acceptable non-toxic acids, including inorganic and organic acids. Such acids include, for example, acetic, benzenesulfonic, benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid and the like. Particularly preferred are citric, hydrobromic, hydrochloric, maleic, phosphoric, sulfuric and tartaric acids.

The pharmaceutical compositions of the present invention comprise an EGFR kinase inhibitor compound and oxaliplatin combination (including pharmaceutically acceptable salts of each component thereof) as active ingredient, a pharmaceutically acceptable carrier and optionally other therapeutic ingredients or adjuvants. Other therapeutic agents may include those cytotoxic, chemotherapeutic or anti-cancer agents, or agents which enhance the effects of such agents, as listed above. The compositions include compositions suitable for oral, rectal, topical, and parenteral (including subcutaneous, intramuscular, and intravenous) administration, although the most suitable route in any given case will depend on the particular host, and nature and severity of the conditions for which the active ingredient is being administered. The pharmaceutical compositions may be conveniently presented in unit dosage form and prepared by any of the methods well known in the art of pharmacy.

In practice, the compounds represented by an EGFR kinase inhibitor compound and oxaliplatin combination (including pharmaceutically acceptable salts of each component thereof) of this invention can be combined as the active ingredient in intimate admixture with a pharmaceutical carrier according to conventional pharmaceutical compounding techniques. The carrier may take a wide variety of forms depending on the form of preparation desired for administration, e.g. oral or parenteral (including intravenous). Thus, the pharmaceutical compositions of the present invention can be presented as discrete units suitable for oral administration such as capsules, cachets or tablets each containing a predetermined amount of the active ingredient. Further, the compositions can be presented as a powder, as granules, as a solution, as a suspension in an aqueous liquid, as a non-aqueous liquid, as an oil-in-water emulsion, or as a water-in-oil liquid emulsion. In addition to the common dosage forms set out above, an EGFR kinase inhibitor compound and oxaliplatin combination (including pharmaceutically acceptable salts of each component thereof) may also be administered by controlled release means and/or delivery devices. The combination compositions may be prepared by any of the methods of pharmacy. In general, such methods include a step of bringing into association the active ingredients with the carrier that constitutes one or more necessary ingredients. In general, the compositions are prepared by uniformly and intimately admixing the active ingredient with liquid carriers or finely divided solid carriers or both. The product can then be conveniently shaped into the desired presentation.

Thus, the pharmaceutical compositions of this invention may include a pharmaceutically acceptable carrier and an EGFR kinase inhibitor compound and oxaliplatin combination (including pharmaceutically acceptable salts of each component thereof). An EGFR kinase inhibitor compound and oxaliplatin combination (including pharmaceutically acceptable salts of each component thereof), can also be included in pharmaceutical compositions in combination with one or more other therapeutically active compounds. Other therapeutically active compounds may include those cytotoxic, chemotherapeutic or anti-cancer agents, or agents which enhance the effects of such agents, as listed above.

Thus in one embodiment of this invention, a pharmaceutical composition can comprise an EGFR kinase inhibitor compound and oxaliplatin in combination with an anticancer agent, wherein said anti-cancer agent is a member selected from the group consisting of alkylating drugs, antimetabolites, microtubule inhibitors, podophyllotoxins, antibiotics, nitrosoureas, hormone therapies, kinase inhibitors, activators of tumor cell apoptosis, and antiangiogenic agents.

The pharmaceutical carrier employed can be, for example, a solid, liquid, or gas. Examples of solid carriers include lactose, terra alba, sucrose, talc, gelatin, agar, pectin, acacia, magnesium stearate, and stearic acid. Examples of liquid carriers are sugar syrup, peanut oil, olive oil, and water. Examples of gaseous carriers include carbon dioxide and nitrogen.

In preparing the compositions for oral dosage form, any convenient pharmaceutical media may be employed. For example, water, glycols, oils, alcohols, flavoring agents, preservatives, coloring agents, and the like may be used to form oral liquid preparations such as suspensions, elixirs and solutions; while carriers such as starches, sugars, microcrystalline cellulose, diluents, granulating agents, lubricants, binders, disintegrating agents, and the like may be used to form oral solid preparations such as powders, capsules and tablets. Because of their ease of administration, tablets and capsules are the preferred oral dosage units whereby solid pharmaceutical carriers are employed. Optionally, tablets may be coated by standard aqueous or nonaqueous techniques.

A tablet containing the composition of this invention may be prepared by compression or molding, optionally with one or more accessory ingredients or adjuvants. Compressed tablets may be prepared by compressing, in a suitable machine, the active ingredient in a free-flowing form such as powder or granules, optionally mixed with a binder, lubricant, inert diluent, surface active or dispersing agent. Molded tablets may be made by molding in a suitable machine, a mixture of the powdered compound moistened with an inert liquid diluent. Each tablet preferably contains from about 0.05 mg to about 5 g of the active ingredient and each cachet or capsule preferably containing from about 0.05 mg to about 5 g of the active ingredient.

For example, a formulation intended for the oral administration to humans may contain from about 0.5 mg to about 5 g of active agent, compounded with an appropriate and convenient amount of carrier material that may vary from about 5 to about 95 percent of the total composition. Unit dosage forms will generally contain between from about 1 mg to about 2 g of the active ingredient, typically 25 mg, 50 mg, 100 mg, 200 mg, 300 mg, 400 mg, 500 mg, 600 mg, 800 mg, or 1000 mg.

Pharmaceutical compositions of the present invention suitable for parenteral administration may be prepared as solutions or suspensions of the active compounds in water. A suitable surfactant can be included such as, for example, hydroxypropylcellulose. Dispersions can also be prepared in glycerol, liquid polyethylene glycols, and mixtures thereof in oils. Further, a preservative can be included to prevent the detrimental growth of microorganisms.

Pharmaceutical compositions of the present invention suitable for injectable use include sterile aqueous solutions or dispersions. Furthermore, the compositions can be in the form of sterile powders for the extemporaneous preparation of such sterile injectable solutions or dispersions. In all cases, the final injectable form must be sterile and must be effectively fluid for easy syringability. The pharmaceutical compositions must be stable under the conditions of manufacture and storage; thus, preferably should be preserved against the contaminating action of microorganisms such as bacteria and fungi. The carrier can be a solvent or dispersion medium containing, for example, water, ethanol, polyol (e.g., glycerol, propylene glycol and liquid polyethylene glycol), vegetable oils, and suitable mixtures thereof.

Pharmaceutical compositions of the present invention can be in a form suitable for topical sue such as, for example, an aerosol, cream, ointment, lotion, dusting powder, or the like. Further, the compositions can be in a form suitable for use in transdermal devices. These formulations may be prepared, utilizing an EGFR kinase inhibitor compound and oxaliplatin combination (including pharmaceutically acceptable salts of each component thereof) of this invention, via conventional processing methods. As an example, a cream or ointment is prepared by admixing hydrophilic material and water, together with about 5 wt % to about 10 wt % of the compound, to produce a cream or ointment having a desired consistency.

Pharmaceutical compositions of this invention can be in a form suitable for rectal administration wherein the carrier is a solid. It is preferable that the mixture forms unit dose suppositories. Suitable carriers include cocoa butter and other materials commonly used in the art. The suppositories may be conveniently formed by first admixing the composition with the softened or melted carrier(s) followed by chilling and shaping in molds.

In addition to the aforementioned carrier ingredients, the pharmaceutical formulations described above may include, as appropriate, one or more additional carrier ingredients such as diluents, buffers, flavoring agents, binders, surface-active agents, thickeners, lubricants, preservatives (including anti-oxidants) and the like. Furthermore, other adjuvants can be included to render the formulation isotonic with the blood of the intended recipient. Compositions containing an EGFR kinase inhibitor compound and oxaliplatin combination (including pharmaceutically acceptable salts of each component thereof) may also be prepared in powder or liquid concentrate form.

Dosage levels for the compounds of the combination of this invention will be approximately as described herein, or as described in the art for these compounds. It is understood, however, that the specific dose level for any particular patient will depend upon a variety of factors including the age, body weight, general health, sex, diet, time of administration, route of administration, rate of excretion, drug combination and the severity of the particular disease undergoing therapy.

This invention will be better understood from the Experimental Details that follow. However, one skilled in the art will readily appreciate that the specific methods and results discussed are merely illustrative of the invention as described more fully in the claims which follow thereafter, and are not to be considered in any way limited thereto.

Experimental Details:

Summary and Conclusions

Erlotinib (Tarceva™, OSI-774) is a potent, orally bioavailable, small molecule inhibitor of EGFR (HER1, erbB1) tyrosine kinase (TK). Erlotinib inhibits phosphorylation of the EGFR tyrosine kinase domain, thereby blocking key signal transduction molecules downstream from the receptor. Erlotinib is being tested in Phase III clinical trials in NSCLC and is also being tested in other types of solid tumors. Oxaliplatin is used in the management of patients with advanced colorectal cancer. In our studies, the anti-tumor activity of erlotinib has been evaluated in two human colorectal tumor xenograft models (LoVo and HCT116) in athymic mice. Both cell types express EGFR and have a similar doubling time in vitro and in vivo. Erlotinib was administered as monotherapy or in combination with oxaliplatin to mice with established LoVo or HCT116 tumors. Drugs were combined at their respective maximum therapeutic dose or at suboptimal doses.

In the LoVo model, treatment of mice with erlotinib at 100 mg/kg resulted in profound tumor growth inhibition (TGI>100%, p<0.001), with 6/10 mice showing partial regressions (PR). At 25 mg/kg, erlotinib treatment caused significant tumor growth inhibition (TGI=79%, p<0.001). Oxaliplatin administered at 10 mg/kg or 5 mg/kg showed marginal anti-tumor activity in the LoVo model (TGI=39%, p=0.056; TGI=33%, p=0.06). The combination of erlotinib and oxaliplatin at their respective maximum therapeutic doses resulted in enhanced anti-tumor activity (TGI>100%, p<0.001) in the vast majority of treated mice. This enhanced activity was superior to activity of single agents. Co-administration of erlotinib/oxaliplatin at suboptimal doses, resulted in enhanced anti-tumor activity (TGI=75%) compared with monotherapy activity.

Oxaliplatin administered at 10 mg/kg or 5 mg/kg showed marginal anti-tumor activity in the HCT116 model (TGI=26%, p=0.307; TGI=21%, p=0.273, respectively; data not shown). This is similar to our findings in the LoVo model and has led us to question whether this literature derived dose and regimen is optimal. The combination of erlotinib and oxaliplatin at either their respective maximum therapeutic doses or suboptimal doses did nothing to enhance anti-tumor activity against the HCT116 tumor model in treated mice (data not shown). Thus, data for combining oxaliplatin and Tarceva™ may vary based on differences in the molecular biology of given colorectal cancer tumor lines or tumors.

Together, the data support the conclusion that erlotinib, especially at suboptimal doses, can enhance the anti-tumor activity of oxaliplatin, without enhanced toxicity, in a human colorectal tumor xenograft model. These data support the clinical evaluation of erlotinib in human colorectal cancer.

Glossary of Abbreviations
Bwl Body weight loss
CMC Carboxymethylcellulose
EGFR Epidermal growth factor receptor
EGFRi Epidermal growth factor receptor inhibitor
ip intraperitoneal
iv intravenous
MTD Maximum tolerated dose
NSCLC Non-small cell lung cancer
q3d dosing every three days
q4d dosing every four days
q6d dosing every six days
q7d dosing every seven days
qd once daily (dosing)
po oral
PBS Phosphate Buffered Saline
SEM Standard error of the mean
TGI Tumor Growth Inhibition

Materials and Methods

The goal of this study is to assess the anti-tumor efficacy of the small molecule epidermal growth factor receptor inhibitor (EGFRi) Tarceva™ in combination with oxalipaltin on LoVo colorectal human xenograft, grown in female athymic nu/nu mice. Oxaliplatin is an agent that is currently used clinically in the treatment of colorectal cancer alone and in combination with other chemotherapeutic agents and/or radiation, depending on the stage of disease. In this study, the drugs were combined at their respective maximum tolerated dose (MTD) and also combined together at sub-optimal doses. All doses included in the combination groups were also included in the study as monotherapy arms. The attempt was to achieve maximum efficacy/regression without increased toxicity.

Animals

Female nude mice (10/group), obtained from Charles River Laboratories (Wilmington, Mass.) at an age of 4-6 weeks, and were used when they were ˜10-12 weeks old and weighed ˜23-25 grams. The health of all animals was determined daily by gross observation of experimental animals and by the analyses of blood samples of sentinel animals that were housed on the shared shelf racks. All animals were allowed to acclimate and recover from any shipping related stress for one week prior to experimental manipulation. Autoclaved water and irradiated food (5058-ms Pico chow (mouse) Purina, Richmond, Ind.) were provided ad libitum, and the animals were maintained in a 12 hour light and dark cycle. Cages, bedding and water bottles were autoclaved before use and were changed weekly. The mice are housed at 10-12 animals per polycarbonate cage (17.5×9×6 inches) with Certified BetaChip bedding (Northeastern Products, Warrensburg, N.Y.). All in vivo experiments were performed in accordance with protocols approved by the Roche Animal Care and Use Committee (RACUC). The Roche animal care facility is fully accredited by the American Association for the Accreditation of Lab Animal Care (AAALAC).

Tumors

LoVo cells were grown in F-12K+20% FBS (not heat inactivated) and harvested. 5×10⁶ cells/0.2 ml/mouse in PBS (Phosphate Buffered Saline) were implanted subcutaneously in the right flank on Aug. 16, 2002 for efficacy study 540.

Test Agent

Tarceva™ for study 540 was formulated as a suspension (12.5 or 3.125 mg/ml) in sodium carboxymethylcellulose (CMC)-7L2 (3 mg/ml) and Tween 80 (1 mg/ml) in sterile water for injection. Formulated compound was made up in one batch for the entire 3 week study.

Oxaliplatin was provided in clinical form. It was formulated in the prepackaged vial with 5% dextrose according to the label instructions, rendering a solution containing 5 mg/ml active compound. An aliquot of the stock vial solutions was taken for each dose group representing the drug needed for that group for the entire study duration and diluted out further with sterile saline, to provide a solution that renders a 0.2 ml dosing volume for each individual animal.

Randomization

For study 540, animals were randomized according to tumor volume on day 17 post tumor implantation so that all groups had similar starting mean tumor volumes of 100-150 mm³.

Study Designs

The design of the study is shown in Table 1.

TABLE 1
Dose groups for LoVo efficacy 540 study with Tarceva ™ and
Oxaliplatin

| | | Dose | | |
|---|---|---|---|---|
| Group | Treatment | (mg/kg) | Frequency | Route |
| 1 | Oral Vehicle/IP Vehicle | 0 | qd/q3d | Oral/IP |
| 2 | Tarceva ™ | 100 | qd | Oral |
| 3 | Tarceva ™ | 25 | qd | Oral |
| 4 | Oxaliplatin | 10 | q7d | IP |
| 5 | Oxaliplatin | 5 | q7d | IP |
| 6 | Tarceva ™/Oxaliplatin | 100/10 | qd/q7d | Oral/IP |
| 7 | Tarceva ™/Oxaliplatin | 25/5 | qd/q7d | Oral/IP |

Treatment

For efficacy study 540, treatment began on Sep. 5, 2002 (Day 18 post-tumor implant). Tarceva™ was administered using a 1 cc syringe and 18-gauge gavage needles (0.2 ml/animal). Oxaliplatin was administered ip using a 1-cc syringe and 26-gauge needle (0.2 ml/animal). All groups were treated q7d for 3 weeks (total 3 injections). Treatment ended on Sep. 23, 2002 (Day 39 post-tumor implant) for all animals. No end of study drug exposure analysis was performed on this study.

Pathology/Necropsy

A full necropsy was performed on five mice per treatment from all remaining groups. Whole blood was also collected for hematology and clinical chemistry. Tumors were removed and fixed in zinc formalin and subsequently embedded in paraffin. Immunohistochemistry can be performed on these sections to assess apoptosis via TUNEL and proliferative index via Ki67. Necrosis can also be assessed on H&E stained sections.

Monitoring

Tumor measurements and mouse weights were taken two-three times per week for LoVo. All animals were individually followed throughout the experiment.

Calculations & Statistical Analysis

Weight loss was graphically represented as percent change in mean group body weight, using the formula:
((W−W0)/W0)×100,
where ‘W’ represents mean body weight of the treated group at a particular day, and ‘W0’ represents mean body weight of the same treated group at initiation of treatment. Maximum weight loss was also represented using the above formula, and indicated the maximum percent body weight loss that was observed at any time during the entire experiment for a particular group.

Efficacy data was graphically represented as the mean tumor volume+standard error of the mean (SEM). Tumor volumes of treated groups were presented as percentages of tumor volumes of the control groups (% T/C), using the formula:
100×((T−T0)/(C−C0)),
where T represented mean tumor volume of a treated group on a specific day during the experiment, T0 represented mean tumor volume of the same treated group on the first day of treatment; C represented mean tumor volume of a control group on the specific day during the experiment, and C0 represented mean tumor volume of the same treated group on the first day of treatment.

Tumor volume (in cubic millimeters) was calculated using the ellipsoid formula:
(D×(d2))/2
where ‘D’ represents the large diameter of the tumor, and ‘d’ represents the small diameter.

In some cases, tumor regression and/or percent change in tumor volume was calculated using the formula:
((T−T0)/T0)×100
where ‘T’ represents mean tumor volume of the treated group at a particular day and ‘T0’ represents mean tumor volume of the same treated group at initiation of treatment.

Statistical analysis was determined by the rank sum test and One Way Anova and a post-hoc Bonferroni t-test (SigmaStat, version 2.0, Jandel Scientific, San Francisco, Calif.). Differences between groups were considered to be significant when the probability value (p) was <0.05.

Results and Discussion

Results

Toxicity

Unscheduled Deaths

Tarceva™ and Oxaliplatin—Experiment 540.

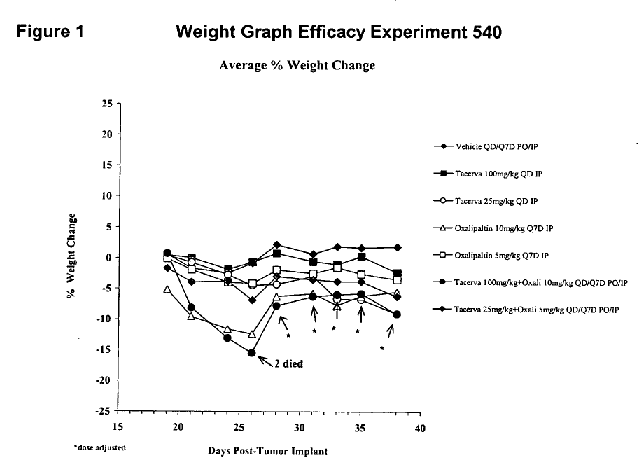

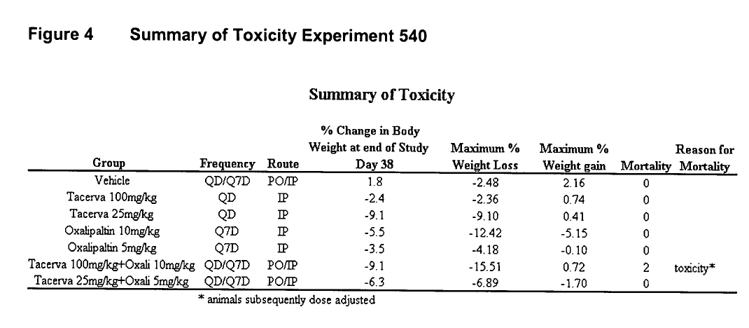

On day 28 post tumor implantation, mouse # 4 and & # 7 from the Tarceva™ 100 mg/kg, Oxalipaltin. Both had around 20% body weight loss (FIGS. 1 and 4). No overt findings at necropsy.

Weight Changes and Clinical Signs

Tarceva™ and Oxaliplatin Experiment 540

Clear toxicity was evident in the Tarceva™ 100 mg/kg, Oxaliplatin 10 mg/kg combination (Group 6) very early in the study, with an average weight loss of −15% and a severe reddening of the skin after five to seven days of treatment (day's 22-30 post tumor implantation) (FIGS. 1 and 4). By day 25 post tumor implantation, this group had an average body weight loss of 15%. Two animals died on day 26 post tumor implantation. Animals were subsequently dose adjusted for the remainder of the study.

Mice treated with 100 mg/kg Tarceva™ (Group 2) presented with the classic reddening of the skin as seen in several past studies. No other signs of toxicity were noted in any other dose groups as assessed by measuring changes in body weight and gross observation of individual animals (FIGS. 1 and 4).

Efficacy

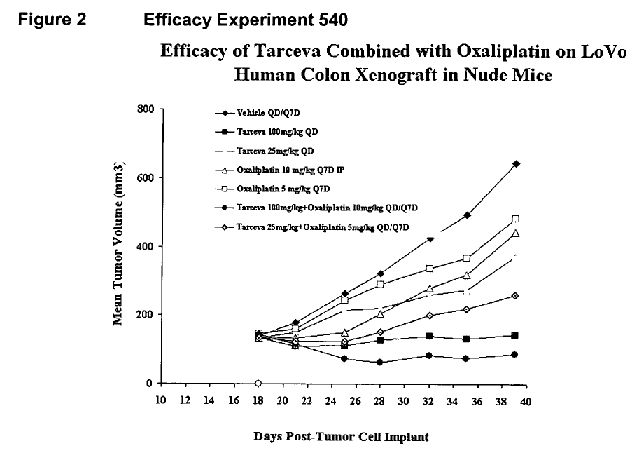

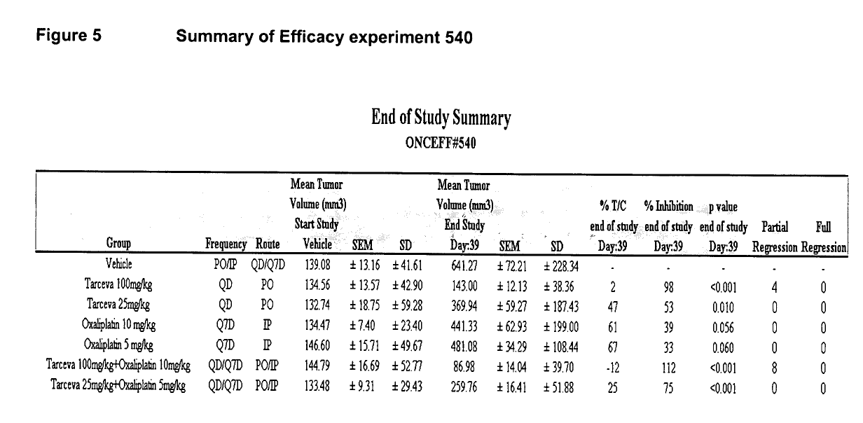

Tarceva™ and Oxaliplatin Experiment 540 (FIGS. 2 and 5)

At study termination (day 39 post tumor implant, treatment day 19) significant tumor efficacy against the LoVo colorectal xenograft was seen again with Tarceva™ monotherapy at 100 mg/kg qd (98%, % T/C=2%, P=<0.001) with four partial regressions (16% and 7%). The sub-optimal single agent low dose of 25 mg/kg qd Tarceva™ also rendered similar activity to past studies showing 53% (% T/C=47%) tumor growth inhibition in this study.

Oxaliplatin was tested at two monotherapy doses in this study. No significant tumor growth inhibition was seen at the dose of 10 mg/kg q7d ip of Oxaliplatin (39%, % T/C=61%, P=0.056). At the sub-optimal dose 5 mg/kg q7d ip, only 33% tumor growth inhibition was seen (% T/C=67%).

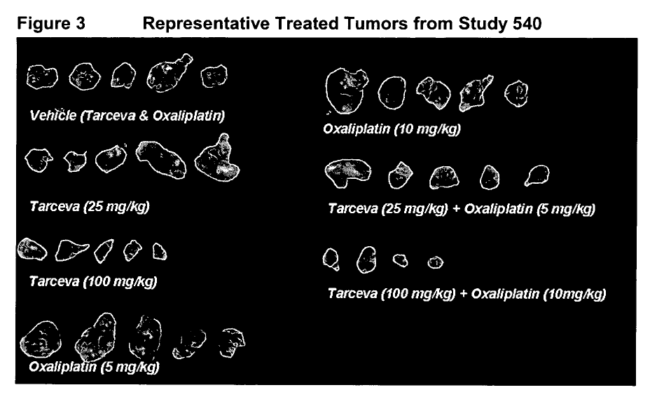

Combinations of oxaliplatin and Tarceva™ were assessed in the LoVo colorectal xenograft to see if antagonistic, additive or synergistic activity would prevail. Oxaliplatin and Tarceva™ were combined at the high doses of 10 mg/kg q7d ip and 100 mg/kg qd po, respectively although toxicity appeared as early as 3 days post study initiation, and two mouse deaths were noted, with dose adjusting, the majority of mice in the group survived. Significant tumor growth inhibition was seen in this combination group (>100%, % T/C=−12%, P=<0.001) with 80% of the tumors partially regressed, with no complete regressions noted. This tumor growth inhibition could be classified as synergistic being significantly better than both high dose oxaliplatin (P<0.001) and Tarceva™ (P=0.002). The sub-optimal doses of oxaliplatin at 5 mg/kg q7d ip and Tarceva™ 25 mg/kg qd po were also combined. This combination was well tolerated by all mice rendering moderate weight loss which recovered later. Significant tumor growth inhibition superior to vehicle control was seen (75%, % T/C=25%, P=<0.001). This tumor growth inhibition could be classified as synergistic being significantly better than sub-optimal oxaliplatin (P <0.01) and sub-optimal Tarceva™ (P<0.01). Representative tumors from treated mice are shown in FIG. 3.

Discussion

Recently, the EGFR has emerged as a key target for anticancer therapeutics. Tarceva™ is an orally active, selective epidermal growth factor receptor-inhibtor, which blocks signal transaction pathways implicated in proliferation and survival of cancer cells, and is in phase III clinical trial. In the present study, we evaluated the combined use of Tarceva™ with classic chemotherapy drug by using the LoVo human colorectal xenograft model. Lovo tumor model represents a colorectal cancer which expresses EGFR, and therefore is likely to respond to an epidermal growth factor receptor-inhibtor (Magne N, et al. (2002) Br. J. Cancer 86(9):1518-1523).

Human colorectal cancer represents one of the most prevalent human carcinomas. Surgical resection is the only curative treatment. Since the majority of patients present in an advanced stage of disease with metastatic spread, surgery alone is not a good enough clinical approach. Newer treatments are being sought to better manage this disease. Ideally these would come in the form of new single agent entities. The trend for novel agents, however, is to pursue targets inherent only to the cancer cells. With this precise targeting comes the assumption of a better toxicity profile compared to traditional cytotoxic agents.

Initial studies in vivo demonstrated clear antitumor effect in a wide spectrum of cancer models including non-small cell lung cancer (NSCLC), colorectal cancer, breast cancer, and others. In the current studies, the novel EGFR inhibitor Tarceva™ was combined in a dual fashion with clinically relevant chemotherapeutic agents in the LoVo xenograft model. The agents were combined at their MTD's to represent the most intensive clinical regimen. A combination of sub-optimal doses representing ¼ MTD for Tarceva™+chemotherapy was also assessed to look for potentiated efficacy or perhaps antagonism.

Many traditional cytotoxics have single-agent activity in colorectal cancer including CPT-11, taxol, 5-flourouracil, and oxaliplatin. Oxaliplatin was recently approved for clinical use in Europe, hence inclusion of this agent in our studies here. Since only modest objective responses were seen with monotherapy regimens, combination approach is considered a better approach. The ideal regimen would be two agents with different mechanisms which could therefore potentially achieve synergic or additive efficacy with toxicity reduced or similar to monotherapy treatment. Epidermal growth factor receptor-inhibitor seems to have the promising perspective for achieving this goal when combined with traditional chemotherapeutics.

Several EGFR inhibitors are in the later stages of clinical development. Two antibodies against EGFR have been developed. Cetuximab (C225, Erbitux), a chimeric antibody which competitively inhibits the activation of EGFR, and ABX-EGF, a fully humanized antibody to EGFR that is postulated to escape degradation post-internalization and therefore gets recycled. Impressive clinical results have been seen with Cetuximab, and Phase II results from ABX-EGF are pending. Several small molecules are also in development. Of particular interest are Iressa™ (ZD1839), C₁₋₁₀₃₃ and Tarceva™ (OSI-774). CI-1033, being earliest in development, is a nonspecific irreversible inhibitor of all EGFR family members. Data from later stage trials with this compound are pending. Iressa™ received FDA approval as third line treatment for NSCLC in May 2003.

Preliminary studies were performed in naïve female nude mice to determine the maximum tolerated dose (MTD) of Tarceva™. The MTD was defined as a dose that renders <20% body weight loss and no death in a 14 day study. An MTD for Tarceva™ in the CMC/Tween formulation in a MTD study was 100 mg/kg qd, with 200 mg/kg qd showing toxicity. However, our previous efficacy studies have also shown that 150 mg/kg of Tarceva™ given once a day in CMC/Tween is tolerated well for 3 weeks. Based on literature, a dosage for oxaliplatin of 10 mg/kg, once a week intraperitoneal was adopted in the study (Cividalli A, et al. (2002) Int. J. Radiat. Oncol. Biol. Phys. 52(4):1092-1098).

In the current studies, the novel EGFR inhibitor Tarceva™ was combined with the clinically relevant chemotherapeutic agent oxaliplatin in the LoVo colorectal xenograft model. Oxaliplatin was combined at the maximum therapeutic dose to represent the most intensive clinical regimen. A combination of sub-optimal doses of Tarceva™+oxaliplatin was also assessed to look for potentiated efficacy or perhaps antagonism.

The data clearly shows impressive single agent activity of each agent in the LoVo colorectal human tumor xenograft at their respective maximum therapeutic dose (Tarceva™ 100 mg/kg >100% TGI, p<0.001, 98%, TGI, p<0.001 (experiment 525 and 540, respectively)) with 40-60% of the tumors partially regressed. The sub-optimal single agent low dose of Tarceva™ (25 mg/kg qd) showed around 53-79% tumor growth inhibition.

Combinations of Tarceva™ and oxaliplatin were also assessed in the LoVo colorectal xenograft to see if antagonistic, additive or synergistic activity would prevail. Oxaliplatin and Tarceva™ were combined at the high doses of 10 mg/kg q7d ip and 100 mg/kg qd po and lower doses of 5 mg/kg q7d ip oxaliplatin and Tarceva™ 25 mg/kg, respectively. Although toxicity appeared as early as 5 days post study initiation, with two mouse deaths, with dose adjusting, the majority of mice in the group survived. Significant tumor growth inhibition was seen in the high dose combination group (>100%, % T/C=−12%, P=<0.001) with 80% of the tumor partial regression. This tumor growth inhibition could be classified as synergistic being significantly better than both single agent high dose oxaliplatin (P<0.001) and Tarceva™ (P<0.001). The sub-optimal doses of oxaliplatin at 5 mg/kg q7d iv and Tarceva™ 25 mg/kg qd po were also combined. This combination was well tolerated by all mice rendering only mild weight loss or gross signs of toxicity. Significant tumor growth inhibition superior to vehicle control was seen (75%, % T/C=25%, P >0.001) . . . This tumor growth inhibition could be classified as synergistic being significantly better than both sub-optimal doses of oxaliplatin (P=0.009) and Tarceva™ (P<0.001).

### CONCLUSION

Erlotnib (Tarceva™, OSI-774) is a potent, orally bioavailable, small molecule inhibitor of EGFR (HER1, erbB1) tyrosine kinase (TK). Erlotinib inhibits phosphorylation of the EGFR tyrosine kinase domain, thereby blocking key signal transduction molecules downstream from the receptor. Erlotinib is completing Phase III trials in NSCLC and is also being tested in other types of solid tumors. Oxaliplatin is used in the management of patients with advanced colorectal cancer. In our studies, the anti-tumor activity of erlotinib has been evaluated in two human colorectal tumor xenograft models (LoVo and HCT116) in athymic mice. Both cell types express EGFR and have a similar doubling time in vitro and in vivo. Erlotinib was administered as monotherapy or in combination with oxaliplatin to mice with established LoVo or HCT116 tumors. Drugs were combined at their respective maximum therapeutic dose or at suboptimal doses. In the LoVo model, treatment of mice with erlotinib at 100 mg/kg resulted in profound tumor growth inhibition (TGI>100%, p<0.001), with 6/10 mice showing partial regressions (PR). At 25 mg/kg, erlotinib treatment caused significant tumor growth inhibition (TGI=79%, p<0.001). Oxaliplatin administered at 10 mg/kg or 5 mg/kg showed marginal anti-tumor activity in the LoVo model (TGI=39%, p=0.056; TGI=33%, p=0.06). The combination of erlotinib and oxaliplatin at their respective maximum therapeutic doses resulted in enhanced anti-tumor activity (TGI>100%, p<0.001) in the vast majority of treated mice. This enhanced activity was superior to activity of single agents. Co-administration of erlotinib and oxaliplatin at suboptimal doses, resulted in enhanced anti-tumor activity (TGI=75%) compared with monotherapy activity.

Oxaliplatin administered at 10 mg/kg or 5 mg/kg showed marginal anti-tumor activity in the HCT116 model (TGI=26%, p=0.307; TGI=21%, p=0.273, respectively; data not shown). This is similar to our findings in the LoVo model and has led us to question whether this literature derived dose and regimen is optimal. The combination of erlotinib and oxaliplatin at either their respective maximum therapeutic doses or suboptimal doses did nothing to enhance anti-tumor activity against the HCT116 tumor model in treated mice (data not shown). However, data for combining oxaliplatin and Tarceva™ may vary based on differences in the molecular biology of given colorectal cancer tumor lines.

Together, the data support the conclusion that erlotinib, especially at suboptimal doses, can enhance the anti-tumor activity of oxaliplatin, without enhanced toxicity, in human colorectal tumor xenograft models. These data support the evaluation of erlotinib in human colorectal cancer.

Incorporation by Reference

All patents, published patent applications and other references disclosed herein are hereby expressly incorporated herein by reference.

Equivalents

Those skilled in the art will recognize, or be able to ascertain, using no more than routine experimentation, many equivalents to specific embodiments of the invention described specifically herein. Such equivalents are intended to be encompassed in the scope of the following claims.

1. A pharmaceutical composition comprising the EGFR kinase inhibitor erlotinib and oxaliplatin, in a pharmaceutically acceptable carrier.

2. The pharmaceutical composition of claim 1, wherein the erlotinib in the composition is present as a hydrochloride salt.

3. The pharmaceutical composition of claim 1, additionally comprising one or more other anti-cancer agents.

4. A composition in accordance with claim 3, wherein said other anti-cancer agent is a member selected from the group consisting of alkylating drugs, antimetabolites, microtubule inhibitors, podophyllotoxins, antibiotics, nitrosoureas, hormone therapies, kinase inhibitors, activators of tumor cell apoptosis, and anti-angiogenic agents.

5. A method for treating tumors or tumor metastases in a patient, comprising administering to said patient simultaneously or sequentially a therapeutically effective amount of the EGFR kinase inhibitor erlotinib and oxaliplatin.

6. The method of claim 5, wherein the patient is a human that is being treated for cancer.

7. The method of claim 5, wherein erlotinib and oxaliplatin are co-administered to the patient in the same formulation.

8. The method of claim 5, wherein erlotinib and oxaliplatin are co-administered to the patient in different formulations.

9. The method of claim 5, wherein erlotinib and oxaliplatin are co-administered to the patient by the same route.

10. The method of claim 5, wherein erlotinib and oxaliplatin are co-administered to the patient by different routes.

11. The method of claim 5, wherein erlotinib is administered to the patient by parenteral or oral administration.

12. The method of claim 5, wherein oxaliplatin is administered to the patient by parenteral administration.

13. The method of claim 5, wherein the tumors or tumor metastases to be treated are selected from lung cancer, colorectal cancer, NSCLC, bronchioloalviolar cell lung cancer, bone cancer, pancreatic cancer, skin cancer, cancer of the head or neck, cutaneous melanoma, intraocular melanoma, uterine cancer, ovarian cancer, rectal cancer, anal region cancer, stomach cancer, gastric cancer, colon cancer, breast cancer, uterine cancer, fallopian tube carcinoma, endometrial carcinoma, cervical carcinoma, vaginal carcinoma, vulval carcinoma, Hodgkin's Disease, esophagus cancer, small intestine cancer, endocrine system cancer, thyroid gland cancer, parathyroid gland cancer, adrenal gland cancer, soft tissue sarcoma, urethral cancer, penis cancer, prostate cancer, bladder cancer, kidney cancer, ureter cancer, renal cell carcinoma, renal pelvis carcinoma, mesothelioma, hepatocellular cancer, biliary cancer, chronic leukemia, acute leukemia, lymphocytic lymphoma, CNS neoplasm, spinal axis cancer, brain stem glioma, glioblastoma multiforme, astrocytoma, schwannoma, ependymoma, medulloblastoma, meningioma, squamous cell carcinoma and pituitary adenoma tumors or tumor metastases.

14. The method of claim 13, wherein the tumors or tumor metastases are refractory.

15. The method of claim 13, wherein the tumors or tumor metastases to be treated are colorectal cancer tumors or tumor metastases.

16. The method of claim 5, additionally comprising administering one or more other anti-cancer agents.

17. The method of claim 16, wherein the other anti-cancer agents are selected from an alkylating agent, cyclophosphamide, chlorambucil, cisplatin, busulfan, irinotecan, melphalan, carmustine, streptozotocin, triethylenemelamine, mitomycin C, an anti-metabolite, methotrexate, etoposide, 6-mercaptopurine, 6-thiocguanine, cytarabine, 5-fluorouracil, capecitabine, dacarbazine, an antibiotic, actinomycin D, doxorubicin, daunorubicin, bleomycin, mithramycin, an alkaloid, vinblastine, paclitaxel, a glucocorticoid, dexamethasone, a corticosteroid, prednisone, a nucleoside enzyme inhibitors, hydroxyurea, an amino acid depleting enzyme, asparaginase, leucovorin, and a folic acid derivative.

18. A method for the treatment of cancer, comprising administering to a subject in need of such treatment (i) a sub-therapeutic first amount of the EGFR kinase inhibitor erlotinib, or a pharmaceutically acceptable salt thereof; and (ii) a sub-therapeutic second amount of oxaliplatin.
